(19) United States
(12) Patent Application Publication
(10) Pub. No.: US 2025/0003903 A1
(43) Pub. Date: Jan. 2, 2025

(54) Device and a System for Detecting Thermal Conductivity of a Fluid

(71) Applicant: ABB Schweiz AG, Baden (CH)

(72) Inventors: Subhashish Dasgupta, Bangalore (IN); Michael May, Bartlesville, OK (US); Jason Lewis, Lewisburg, WV (US); Jason Aaron Lawrence, Owasso, OK (US); Steve Lakey, Bartlesville, OK (US); Steve Hardy, Bixby, OK (US); Max Bailey, Bartlesville, OK (US); Ryan Holsapple, Owasso, OK (US); John Hester, Bartlesville, OK (US)

(73) Assignee: ABB Schweiz AG, Baden (CH)

(21) Appl. No.: 18/886,163

(22) Filed: Sep. 16, 2024

Related U.S. Application Data
(63) Continuation of application No. PCT/IB2023/051144, filed on Feb. 9, 2023.

(30) Foreign Application Priority Data
Mar. 18, 2022 (IN) 202241014954

(51) Int. Cl. G01N 25/18

(52) U.S. Cl. CPC G01N 25/18

(57) ABSTRACT

A thermal conductivity detector device includes a chamber defined with an inlet channel and an outlet channel. The chamber is of an elliptical shape having at least two opposing sides of the chamber defined in an arcuate shape. The chamber is defined in the shape of a venturi. A temperature sensing element is positioned within the chamber. The temperature sensing element is configured to determine the temperature of a fluid flowing through the chamber from the inlet channel towards the outlet channel.

### CROSS-REFERENCE TO RELATED APPLICATIONS

The instant application claims priority to International Patent Application No. PCT/IB2023/051144, filed Feb. 9, 2023, and to Indian patent application Ser. No. 202241014954, filed Mar. 18, 2022, each of which is incorporated herein in its entirety by reference.

### FIELD OF THE DISCLOSURE

The present disclosure generally relates to a thermal conductivity detector device and, more particularly, to a configuration of the thermal conductivity detector device with a plurality of passages that allows flow of fluid, and the fluid impinges a temperature sensing element at multiple surfaces, enhancing detection capabilities of the temperature sensing element.

### BACKGROUND OF THE INVENTION

A thermal conductivity detector device is used for estimating concentration of a particular gas in a mixture of a gases. The device includes an inlet and an outlet with a heat source being positioned between the inlet and the outlet. The heat source may be heated, and its temperature is continuously monitored. Initially gas or fluid of a particular thermal conductivity, is passed to impinge on the heat source. The gas or fluid may cool the heat source and temperature of the heat source is monitored. Subsequently, the gas mixture that is to be analyzed (hereinafter referred to as “the analyte gas”), is passed through the inlet to impinge on the heat source. The temperature of the heat source may increase, or decrease based on the thermal conductivity of the analyte gas. The change in temperature of the heat source is indicated as a thermal signal on an indication device. By estimating the magnitude of change in temperature through the thermal signal on the indication device, the concentration of the analyte gas is estimated.

The accuracy in estimating the concentration of a particular gas is directly dependent on the thermal signal and the sensitivity of the heat source. Therefore, there is a need for improving the accuracy thermal signal in thermal conductivity detector devices.

### BRIEF SUMMARY OF THE INVENTION

In a non-limiting embodiment of the disclosure, a thermal conductivity detector device is disclosed. The device includes a chamber defined with an inlet channel and an outlet channel. The chamber is of an elliptical shape where, at least two opposing sides of the chamber is defined in an arcuate shape and the chamber is defined in the shape of a venturi. A temperature sensing element is positioned within the chamber. The temperature sensing element is configured to determine the temperature of a fluid flowing through the chamber from the inlet channel towards the outlet channel.

In an embodiment of the present disclosure, the arcuate shape of the at least two opposing sides of the chamber is of a pre-determined radius.

In an embodiment of the present disclosure, the chamber is defined by a major axis and a minor axis and, half of sum of the major axis and the minor axis ranges from 0.4 to 0.5.

In an embodiment of the present disclosure, the chamber is defined with a thickness measured along a longitudinal axis of the chamber.

In an embodiment of the present disclosure, the ratio of the thickness to the pre-determined radius ranges from 0.15 to 0.20.

In an embodiment of the present disclosure, each of the inlet channel and the outlet channel are defined on at least one of the same sides of the chamber, two opposing sides of the chamber and two diagonal sides of the chamber.

In an embodiment of the present disclosure, all the sides of the chamber are defined with the arcuate shape.

In an embodiment of the present disclosure, the temperature sensing element is suspended from an inner surface of the chamber.

In an embodiment of the present disclosure, the temperature sensing element is one of a bead with a cylindrical shape, an elliptical shape, a cylindrical shape with a through passage.

In a non-limiting embodiment of the disclosure, a system for estimating concentration of a fluid is disclosed. The system includes a thermal conductivity detector device. The device includes a chamber defined with an inlet channel and an outlet channel. The chamber is of an elliptical shape where, at least two opposing sides of the chamber is defined in an arcuate shape and the chamber is defined in the shape of a venturi. A temperature sensing element is positioned within the chamber. A control unit is communicatively coupled to the temperature sensing element and the control unit is configured to receive a signal from the temperature sensing element corresponding to the temperature of the fluid flowing through the chamber from the inlet channel to the outlet channel. The control unit also indicates the temperature of the fluid through an indication unit where, the temperature of the fluid corresponds to the concentration of the fluid.

In a non-limiting embodiment of the disclosure, a method for estimating concentration of a fluid from a system is disclosed. The method includes the aspect of channeling fluid from an inlet channel to an outlet channel of a chamber. The chamber is of an elliptical shape and at least two opposing sides of the chamber is defined in an arcuate shape, such that the fluid impinges a temperature sensing element positioned in the chamber. The next step involves the aspect of receiving by a control unit, a signal from the temperature sensing element. Further, an indication unit connected to the control unit indicates the temperature of the fluid flowing through the chamber and the temperature of the fluid corresponds to the concentration of the fluid.

### BRIEF DESCRIPTION OF THE SEVERAL VIEWS OF THE DRAWING(S)

FIG. 1 illustrates a sectional perspective view of a thermal conductivity detector device, in accordance with an embodiment of the disclosure.

FIG. 2 illustrates a top perspective view of the thermal conductivity detector device of FIG. 1.

FIG. 3 is a graphical representation showing sensitivity of the thermal signal from the thermal conductivity detector device of FIG. 1 and a conventional thermal conductivity detector device.

FIG. 4 is a graphical representation of the sensitivity of the thermal signal from the thermal conductivity detector device of FIG. 1.

FIG. 5 illustrates a sectional perspective view of the thermal conductivity detector device, in accordance with another embodiment of the disclosure.

FIG. 6 illustrates a side view of the thermal conductivity detector device from the FIG. 5, in accordance with yet another embodiment of the disclosure.

FIG. 7 is a graphical representation of the sensitivity of the thermal signal from the thermal conductivity detector device of FIG. 5 and a conventional thermal conductivity detector device.

FIG. 8 illustrates a sectional perspective view of the thermal conductivity detector device, in accordance with still another embodiment of the disclosure.

FIG. 9 illustrates a side view of the thermal conductivity detector device, in accordance with another embodiment of the disclosure.

FIG. 10 illustrates a side view of the thermal conductivity detector device, in accordance with yet another embodiment of the disclosure.

FIG. 11 illustrates a block diagram of a system for measuring the concentration of a fluid, in accordance with embodiments of the disclosure.

FIG. 12 illustrates a perspective view of a thermal conductivity detector device, in accordance with an embodiment of the disclosure.

FIG. 13 illustrates a sectional perspective view of the thermal conductivity detector device of FIG. 12.

FIG. 14 illustrates another perspective view of the thermal conductivity detector device, in accordance with embodiments of the disclosure.

FIG. 15 illustrates a top view of the thermal conductivity detector device of FIG. 12.

FIG. 16 illustrates a perspective the thermal conductivity detector device, in accordance with another embodiment of the disclosure.

FIG. 17 illustrates a sectional perspective view of the thermal conductivity detector device from the FIG. 16.

FIG. 18 illustrates a top view of the thermal conductivity detector device from the FIG. 16.

FIG. 19 illustrates a top view of the thermal conductivity detector device, in accordance with yet another embodiment of the disclosure.

FIG. 20 is a graphical representation of the sensitivity of the thermal signal from the thermal conductivity detector device of FIG. 12 and a conventional thermal conductivity detector device.

FIG. 21 is a graphical representation of the sensitivity of the thermal signal from the thermal conductivity detector device of FIG. 16 and a conventional thermal conductivity detector device.

FIG. 22 illustrates a perspective view of a temperature sensing element, in accordance with embodiments of the disclosure.

FIG. 23 illustrates a side view of a temperature sensing element of FIG. 22.

FIG. 24 and FIG. 25 illustrates a perspective views temperature sensing element, in accordance with embodiments of the disclosure.

FIG. 26 and FIG. 27 illustrates a side view of the temperature sensing element from the FIG. 24, in accordance with embodiments of the disclosure.

FIG. 28 illustrates a graphical representation of the sensitivity of the temperature sensing element from FIG. 22 and FIG. 24.

FIG. 29 and FIG. 30 illustrates a perspective views the temperature sensing element, in accordance with another embodiment of the disclosure.

FIG. 31 illustrates a side view of the temperature sensing element of the FIG. 29.

FIG. 32 illustrates a perspective view of the temperature sensing element, in accordance with yet another embodiment of the disclosure.

FIG. 33 illustrates a perspective view of the temperature sensing element, in accordance with still another embodiment of the disclosure.

FIG. 34 illustrates a graphical representation of the sensitivity of the temperature sensing element of FIG. 29.

FIG. 35 illustrates a perspective view of the temperature sensing element, in accordance with yet another embodiment of the disclosure.

FIG. 36 illustrates a perspective view of a thermal conductivity detector device, in accordance with an embodiment of the disclosure.

FIG. 37 illustrates a perspective view of a thermal conductivity detector device, in accordance with yet another embodiment of the disclosure.

### DETAILED DESCRIPTION OF THE INVENTION

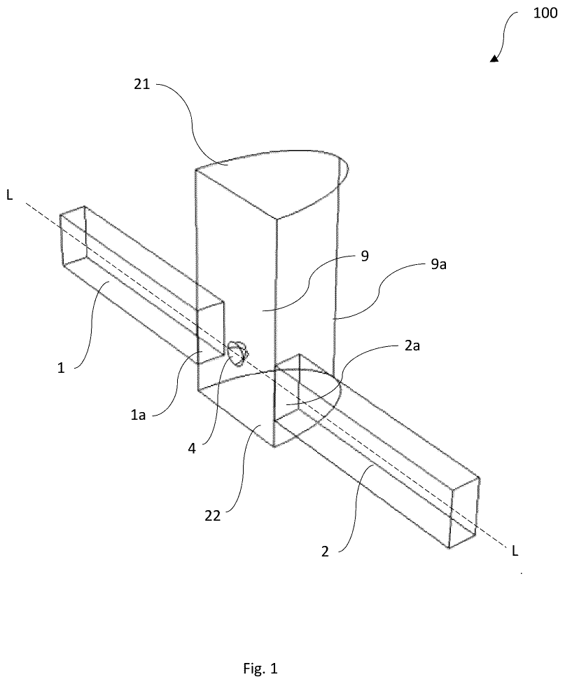

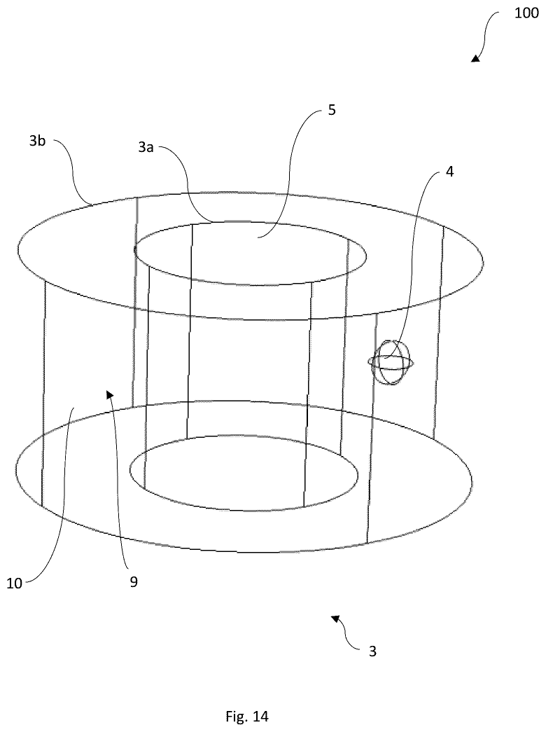

The following paragraphs describe the present disclosure with reference to FIGS. 1 to 37. In the figures, the same element or elements which have similar functions are indicated by the same reference signs. For the purposes of promoting an understanding of the principles of the disclosure, reference will now be made to specific embodiments illustrated in the drawings and specific language will be used to describe the same. It will nevertheless be understood that no limitation of the scope of the disclosure is thereby intended, such alterations and further modifications in the illustrated methods, and such further applications of the principles of the disclosure as illustrated therein being contemplated as would normally occur to one skilled in the art to which the disclosure pertains.

As used in the description below the term “Indication unit” refers to a hardware component which may provide a visual indication or audio or combination of audio-visual indication. As an example, the indication unit may be a computer, a laptop, a tablet, a desktop, a device arranged with Light Emitting Diodes (LED) and the like.

As used in the description below the term “control unit” refers to a hardware component such as at least one processor, volatile memory or nonvolatile memory, software comprising instructions executable by a processor, or a combination of software and hardware. The control unit may include specialized processing units such as integrated system (bus) controllers, memory management control units, floating point units, graphics processing units, digital signal processing units, etc. The processing unit may include a microprocessor, such as AMD Athlon, Duron or Opteron, ARM's application, embedded or secure processors, other line of processors, and the like.

As used in the description, the term “fluid” may be a liquid or a gas. In an embodiment, the term “fluid” may also be implied or refer to a combination of the liquid and the gas. The term “purest form” used in the description is used to indicate the concentration of the fluid. The purest form of the fluid herein indicates the highest concentration of a particular fluid in a fluid mixture.

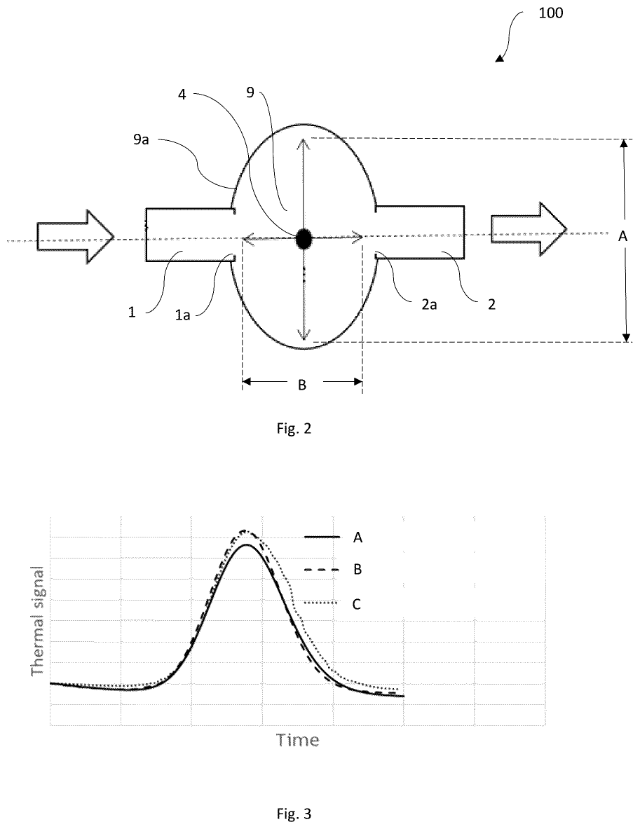

FIG. 1 illustrates a sectional perspective view of a thermal conductivity detector device (100), and FIG. 2 illustrates a top view of the thermal conductivity detector device (100) [hereinafter referred to as the device] of FIG. 1. The device (100) may be defined by a chamber (9), the chamber (9) may include at least one wall (9a) [hereinafter referred to as the wall] and the wall (9a) may be connected at its end to form the chamber (9). The chamber (9) may be of a material with low thermal conductivity. The wall (9a) may further be defined with an inlet port (1a) and an outlet port (2a). The inlet port (1a) of the wall (9a) may be fluidly coupled to an inlet channel (1) and the outlet port (2a) of the wall (9a) may be fluidly coupled to an outlet channel (2). The inlet port (1a) and the outlet port (2a) may be configured to lie diametrically opposite to each other on either side of the wall (9a). Further, the inlet port (1a) and the outlet port (2a) are configured to extend along a lateral axis (L-L) of the wall (9a). The inlet channel (1) and the outlet channel (2) which are fluidly connected to the inlet port (1a) and the outlet port (2a) of the wall (9a) respectively, may also extend along the lateral axis (L-L). The fluid may be directed into the chamber (9) through the inlet channel (1). The fluid is allowed to flow into the chamber (9) from inlet channel (1) and impinges an inner surface [not shown] of the chamber (9). The outlet channel (2) may be configured in a manner such that the fluid that impinges the inner surface of the chamber (9) is received by the outlet channel (2). The fluid that enters the chamber (9) from the inlet channel (1) may be directed out of the chamber (9) by the outlet channel (2).

Further, the device (100) may be an elliptic cylinder with an elliptical cross section. As seen from FIG. 1 and FIG. 2, the device (100) is configured as an elliptic cylinder. The chamber (9) of the device (100) may be enclosed by a top cover (21) and a bottom cover (22). The top cover (21) and the bottom cover (22) may be connected to the wall (9a) of the device (100) to enclose or define the chamber (9). The top cover (21) and the bottom cover (22) may form a roof and a floor of the chamber (9) respectively. In an embodiment, the chamber (9) may not include the top cover (21) and the bottom cover (22), and the chamber (9) may be defined by the wall (9a) in the shape, including but not limited to a sphere or a spherical chamber. The top cover (21) and the bottom cover (22) may be configured to lie diametrically opposite to each other. As seen from FIG. 2, the device (100) may be defined by a major axis (A) and a minor axis (B). The major axis (A) may extend along a direction that is perpendicular to the direction in which the inlet channel (1) and the outlet channel (2) extends. The major axis (A) may extend along a longitudinal direction of the device (100). The major axis (A) may be defined along the direction where the diameter of the device (100) with the elliptical shape is the largest. Further, the minor axis (B) may extend along a direction that is parallel to the direction in which the inlet channel (1) and the outlet channel (2) extends. The minor axis (B) may extend along a lateral direction of the device (100). The minor axis (B) may be defined along the direction where the diameter of the device (100) with the elliptical shape is the smallest. The elliptical shape of the device (100) is defined by half of sum of the major axis (A) and the minor axis (B). The half of sum of the major axis (A) and the minor axis (B) ranges from 0.4 to 0.5 with a tolerance of +/−10%. The elliptical shape of the device (100) may be determined in the above-described manner and the same is illustrated below in an equation that is herein numbered 1.

$$\frac{a + b}{2} = 0.4 to 0.5 with a tolerance of + / - 10 \% \qquad ( equation 1 )$$

The above equation for determining the dimensions of the device (100) with the elliptical shape must not be considered as a limitation since, the parameters of the equation may be varied to obtain a configuration of the device (100) with optimal operational parameters.

The device (100) also includes a temperature sensing element (4) which is positioned inside the chamber (9). The temperature sensing element (4) may be suspended within the chamber (9) and may be positioned at the point in such a manner that the fluid from the inlet channel (1) directly converges onto the temperature sensing element (4). The fluid is directed out of the device (100) via the outlet channel (2) subsequent to the impingement of the fluid on the temperature sensing element (4).

In an embodiment, half of sum of the major axis (A) and the minor axis (B) ranging from 0.4 to 0.5 ensures that the fluid impinges on the temperature sensing element (4) with optimum velocity. Reference is made to FIG. 3 which is a graphical representation of the sensitivity of the thermal signal from the device (100) of FIG. 1 and a conventional thermal conductivity detector device. The curve A represents the thermal signal from the fluid in the conventional device. The curve B represents the thermal signal from the temperature sensing element (4) in the device (100) of the present disclosure where, the half of sum of the major axis (A) and the minor axis (B) is 0.3. Further, the curve C represents the thermal signal from the temperature sensing element (4) in the device (100) of the present disclosure where, the half of sum of the major axis (A) and the minor axis (B) is 0.46. It is evident that the signal sensitivity is significantly greater in the device (100) of the present disclosure. Since the device (100) of the present disclosure is the elliptic cylinder with the elliptical shape, the velocity of fluid impingement on the temperature sensing element (4) is optimized. Further, the above disclosed shape and configuration of the device (100) enhances or increases the overall exposure of the temperature sensing element (4) as the flow of fluid may be directed towards the entire surface of the temperature sensing element (4) from the inlet channel (1), thereby increasing the sensitivity of the temperature sensing element (4). It is further observed from the graph that the optimal results were observed when the device is defined with the elliptical shape where, the half of sum of the major axis (A) and the minor axis (B) is 0.46 as clearly seen from curve C.

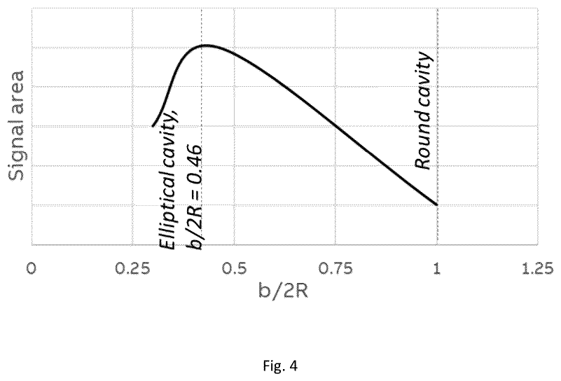

FIG. 4 is a graphical representation of the sensitivity of the thermal signal from the device (100). As again seen from the curve in the FIG. 4, the thermal signal area is significantly higher for the elliptical shape of the chamber (9) where, the half of sum of the major axis (A) and the minor axis (B) is 0.46. However, the thermal signal area is observed to be the lowest for conventional devices where the shape of the chamber (9) is spherical. It is therefore evident that the elliptical shaped chamber (9) enhances the velocity of the fluid impinging on the temperature sensing element (4). It is observed that the velocity of the fluid is neither too high nor is it too low and the velocity is optimized to provide the thermal signal of improved sensitivity in the device (100) of the present disclosure.

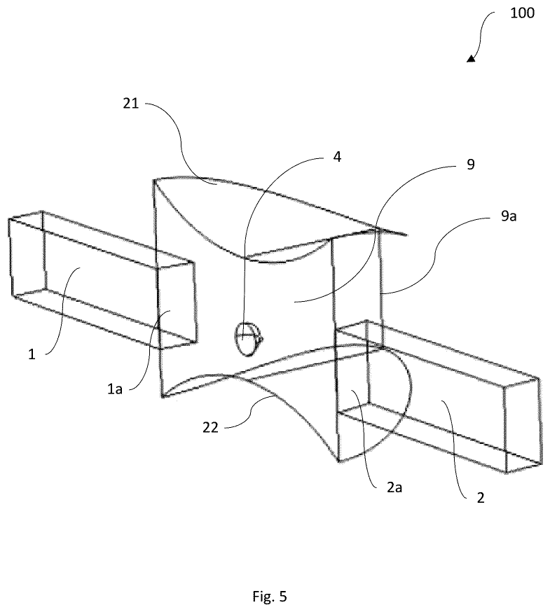

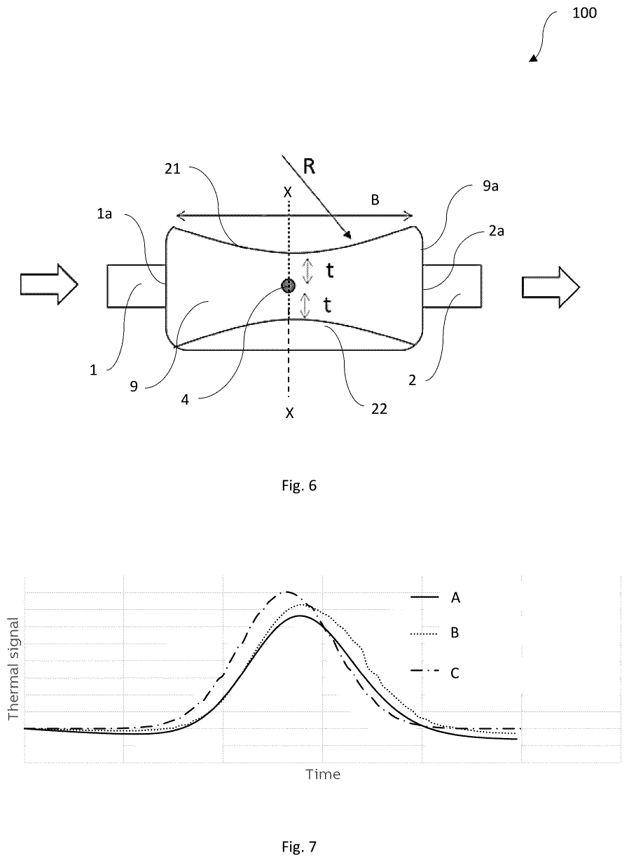

FIG. 5 illustrates a sectional perspective view of an embodiment of the device (100). FIG. 6 illustrates a side view of the embodiment of the device (100) from the FIG. 5. The device (100) may include the chamber (9) which is defined by the wall (9a). The chamber (9) may be enclosed by the top cover (21) and the bottom cover (22) that are connected to the wall (9). The top cover (21) and the bottom cover (22) may be defined with an arcuate shape. The top cover (21) may form the roof of the chamber (9) whereas, the bottom cover (22) may form the floor of the chamber (9). The top cover (21) and the bottom cover (22) in this particular embodiment may be configured diametrically opposite to each other. Further, in this particular embodiment, the top cover (21) and the bottom cover (22) may be defined with the arcuate structure that is of a convex shape. The chamber (9) is in the shape of a venturi with the top cover (21) and the bottom cover (22) being defined by the convex shaped curvature. The convex shaped top cover (21) and the bottom cover (22) may be defined with a pre-determined radius (R) [herein after referred to as the radius]. The radius (R) of the convex shaped top cover (21) and the bottom cover (22) may be measured from a point defined along the longitudinal axis (X-X) of the chamber (9). The chamber (9) may be defined by a pre-determined thickness (t) and the pre-determined thickness (t) of the chamber (9) may be measured along the longitudinal axis (X-X) of the chamber (9). Further, the thickness (t) of the chamber (9) may herein be defined as the distance between the center of the chamber (9) to the tip of the chamber (9) along the longitudinal axis (X-X) of the chamber (9). When the temperature sensing element (4) is positioned in the chamber (9), the thickness (t) of the chamber (9) is measured from the center of the temperature sensing element (4) to the tip of the arcuate shaped top cover (21) or bottom cover (22) along the longitudinal axis (X-X) of the chamber (9). As described above, the venturi shape of the chamber (9) is the half of sum of the major axis (A) and the minor axis (B) ranging from 0.4 to 0.5. Further, the arcuate structure of the top cover (21) and the bottom cover (22) with the convex shape may be defined by the ratio of the thickness (t) to the radius (R) of chamber (9). The ratio of the thickness (t) to the radius (R) of chamber (9) ranges from 0.15 to 0.20 with a tolerance of +/−20%. The dimensions of the convex shape for the top cover (21) and the bottom cover (22) of the chamber (9) may be determined in the above-described manner and the same is illustrated below in an equation that is herein numbered 2.

$$\frac{t}{R} = 0.15 to 0.2 with a tolerance of + / - 20 \% \qquad ( equation 2 )$$

FIG. 7 is a graphical representation of the sensitivity of the thermal signal from the device (100) of FIG. 5 and a conventional thermal conductivity detector device. The curve A represents the thermal signal from the fluid in the conventional device. The curve B represents the thermal signal from the temperature sensing element (4) in the device (100) of the venturi shape where, the half of sum of the major axis (A) and the minor axis (B) is 0.5. Further, the curve C represents the thermal signal from the temperature sensing element (4) in the device (100) with the venturi shape of the chamber (9) and the convex shape on the top cover (21) and the bottom cover (22) of the chamber (9). It is evident that the signal sensitivity is significantly greater in the device (100) of the present disclosure with the venturi shape and the convex shape on the top cover (21) and the bottom cover (22) of the chamber (9). It is also observed that the signal sensitivity for the device (100) with the venturi shape and the convex shape on the top cover (21) and the bottom cover (22) (Curve C) is significantly greater than the signal sensitivity for the device (100) with only the venturi shape (Curve B). The venturi shape along with the convex shape defined to the top cover (21) and the bottom cover (22) optimizes the velocity of fluid impinging on the temperature sensing element (4).

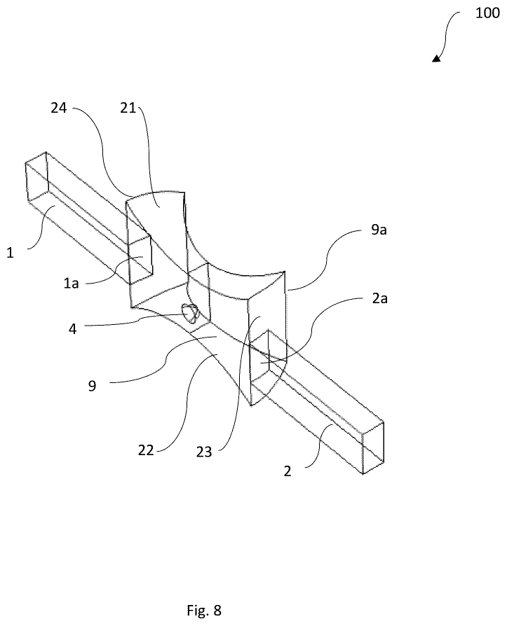

FIG. 8 illustrates a sectional perspective view of another embodiment of the device (100). In this embodiment, the chamber (9) may be defined by the venturi shape where, the half of sum of the major axis (A) and the minor axis (B) is 0.5. Further, the top cover (21) and the bottom cover (22) of the chamber (9) may also be defined by the convex shape as described above. The chamber (9) in this embodiment, may also be defined or enclosed by a front cover (23) and a rear cover (24) which are also configured to lie diametrically opposite to each other. The front cover (23) and the rear cover (24) may also be defined with the convex shape. Further, the chamber (9) in this embodiment may also be defined by the thickness (t) that is measured along the longitudinal axis (X-X) of the chamber (9). The top cover (21), the bottom cover (22), the front cover (23) and the rear cover (24) may be defined by the convex shape with the pre-determined radius (R). The chamber (9) may be defined such that the ratio of the thickness (t) of the chamber (9) to the radius (R) of each of the top cover (21), the bottom cover (22), the front cover (23) and the rear cover (24) ranges from 0.15 to 0.20 with a tolerance of +/−20%. The chamber (9) with the venturi shape and the convex shape of the top cover (21), the bottom cover (22), the front cover (23) and the rear cover (24) impart the shape of a venturi to the chamber (9). Consequently, the fluid is channeled towards the temperature sensing element (4) at greater velocity.

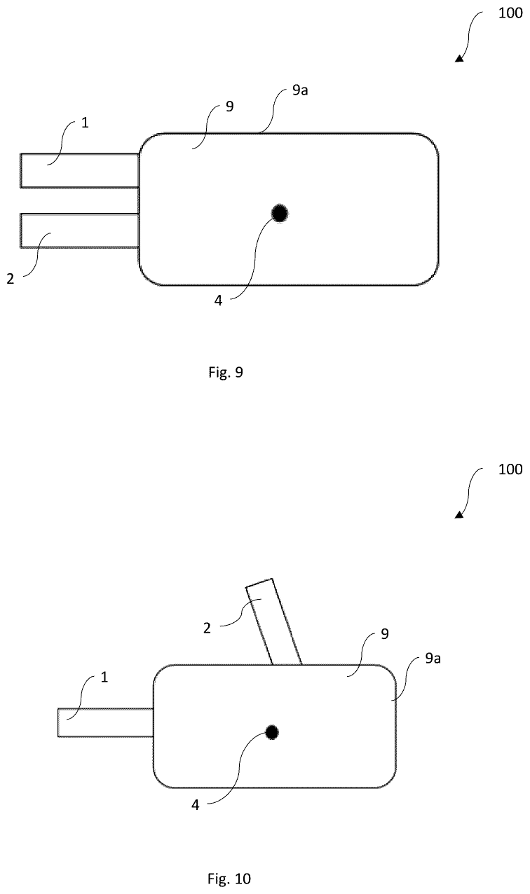

FIG. 9 and FIG. 10 illustrates side views of embodiments of the device (100). As seen from FIG. 9, the inlet channel (1) may be configured to lie on the same side of the chamber (9) as that of the outlet channel (2). The inlet channel (1) may be fluidly coupled to the inlet port (1a) of the chamber (9). The inlet channel (1) may be configured to direct the fluid into the chamber (9) such that the fluid impinges the temperature sensing element (4) accommodated inside the chamber (9). The outlet channel (2) may also be fluidly coupled to the chamber (9) through the outlet port (2a) defined in the chamber (9). The outlet channel (2) may be configured to receive the fluid from the chamber (9) and direct the fluid out of the chamber (9). The outlet channel (2) may be configured adjacent to the inlet channel (1) and may extend parallel to the inlet channel (1). With reference to the FIG. 10, the inlet channel (1) and the outlet channel (2) may be configured to lie perpendicular to each other. The inlet channel (1) may be configured on one side of the chamber (9) whereas, the outlet channel (2) may be configured on the side of the chamber (9) that lies adjacent to the side where the inlet channel (1) is configured. For instance, the inlet channel (1) may be configured to extend towards the chamber (9) from the top cover (21) whereas, the outlet channel (2) may be configured to extend into the chamber (9) from the front cover (23) or the rear cover (24) of the chamber (9).

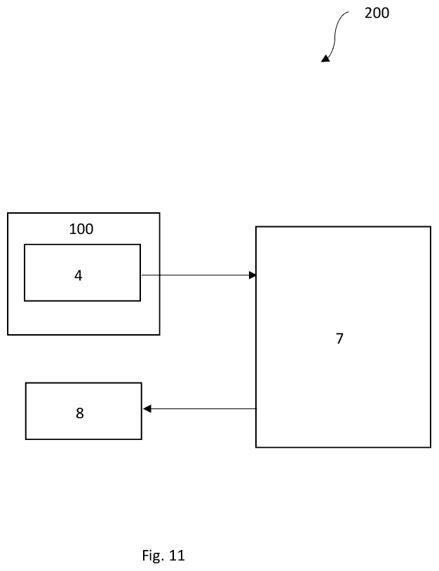

FIG. 11 illustrates a block diagram of a system (200) for measuring the concentration of the fluid. The system (200) may include the control unit (7) that is communicatively coupled to the temperature sensing element (4) in the device (100). The control unit (7) may be configured to send out signals to the temperature sensing element (4) for heating the temperature sensing element (4). The control unit (7) may also be configured to receive signals from the temperature sensing element (4) which correspond to the temperature of the fluid impinging on the temperature sensing element (4). The control unit (7) may also be communicatively coupled to an indication unit (8). The control unit (7) may receive and convert the signals from the temperature sensing element (4) into thermal signals. These thermal signals may further be indicated to a user through the indication unit (8).

Following paragraphs describe working of the device (100) and the system (200) according to the present disclosure. The temperature sensing clement (4) may initially be heated to a first temperature. Subsequently, the fluid may be circulated through the inlet channel (1) and the fluid may flow into the chamber (9) to impinge on the temperature sensing element (4), as seen from FIG. 2 and FIG. 6. The fluid that is initially circulated may herein be referred to as carrier fluid. The carrier fluid may be of the purest form with a high thermal conductivity. Since the thermal conductivity of the carrier fluid is high, the carrier fluid impinges the temperature sensing element (4), and the carrier fluid absorbs the heat from the temperature sensing element (4). The carrier fluid may initially cool the temperature sensing element (4) and the temperature of the temperature sensing element (4) may drop significantly below the initial first temperature to a second temperature. The temperature sensing element (4) may transmit a corresponding signal to the control unit (7). The control unit (7) may convert the received signal into the equivalent thermal signal. The thermal signal may further be indicated to the user through the indication unit (8) and the second temperature may be recorded. Further, an analyte fluid may be circulated to impinge the temperature sensing element (4). The analyte fluid may herein be the fluid whose concentration is to be analyzed or determined. The analyte fluid is a mixture of fluids where the concentration of at least one fluid is to be determined. The analyte fluid passing through the device (100) may be of a higher thermal conductivity or in some instances may be of a lower thermal conductivity. This contrast of thermal conductivity in the fluid is due to the various mixture of fluids. In this exemplary embodiment, the analyte fluid may be of a lower thermal conductivity. Consequently, when the analyte fluid is impinged on the temperature sensing element (4), the analyte fluid does not significantly cool the temperature sensing element (4) due to low thermal conductivity. Therefore, the temperature of the temperature sensing element (4) slightly increases to a third temperature. The third temperature is indicated through the control unit (7) and this increase in temperature directly corresponds to the concentration of the analyte fluid.

In an embodiment, the temperature sensing element (4) may be coupled to a wheat stone bridge for converting the signal received from the temperature sensing element into the corresponding thermal signal.

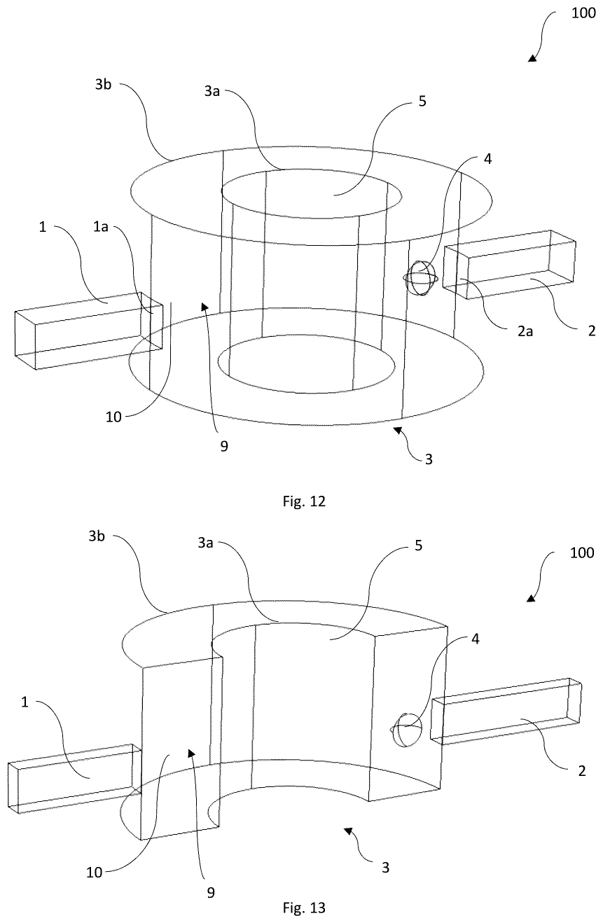

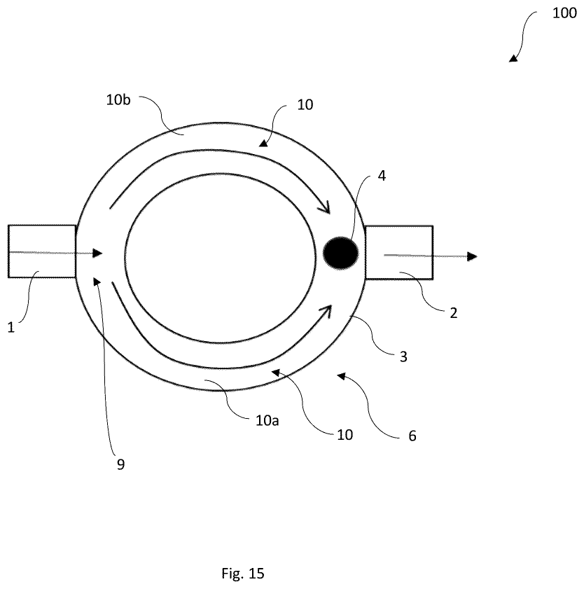

FIG. 12 to FIG. 14 illustrate a perspective view of an embodiment of the device (100). The device (100) may include the chamber (9) and in this particular embodiment, the chamber (9) may be defined by concentric cylinders (3). The concentric cylinders (3) may be configured to facilitate flow of a fluid through the device (100) for measuring the temperature of the fluid. The concentric cylinders (3) include an inner cylinder (3a) and an outer cylinder (3b). The inner cylinder (3a) is concentrically arranged within the outer cylinder (3b) and the diametrical ratio of the inner cylinder (3a) to the outer cylinder (3b) may range from 0.4 to 0.6. The concentric cylinders (3) are constructed such that, a central annular passage (5) is defined at the center of the concentric cylinders (3). With reference to the FIG. 15 which illustrates a top view of the device (100), the configuration of the inner cylinder (3a) and the outer cylinder (3b) may define the chamber (9). The chamber (9) may further define a plurality of passages (10). The fluid may flow through the plurality of passages (10) and the plurality of passages (10) in this embodiment may be categorized as a first passage (10a) and a second passage (10b). In an embodiment, one half of the chamber (9) that is divided along a horizontal axis of the concentric cylinders (3) may define the first passage (10a) whereas the other half of the chamber (9) may define the second passage (10b). The chamber (9) may be fluidly coupled to the inlet channel (1) and the outlet channel (2). The inlet channel (1) and the outlet channel (2) may be fluidly coupled to chamber (9) through the outer cylinder (3b). The inlet channel (1) and the outlet channel (2) may be positioned diametrically opposite to each other. The fluid may be directed into the chamber (9) through the inlet channel (1). The fluid is allowed to flow into the chamber (9) from inlet channel (1) and impinges an inner surface [not shown] of the inner cylinder (3a). The fluid is subsequently divided to flow in two different paths. The fluid may flow through the first passage (10a) and the second passage (10b) of the plurality of passages (10) and converge at a point that lies diametrically opposite to the point at which fluid enters into the chamber (9). The outlet channel (2) may be configured adjacent to the point at which the fluid converges from the first passage (10a) and the second passage (10b).

The device (100) further includes the temperature sensing element (4) which is positioned inside the chamber (9) proximal to the outlet channel (2). The temperature sensing element (4) may be suspended within the chamber (9) and may be positioned at the point where the fluid from the first passage (10a) and the second passage (10b) converges. In an embodiment, the temperature sensing element (4) may be positioned at an annular distance ranging from 50% to 60% of the distance between the inner cylinder (3a) and the outer cylinder (3b). The temperature sensing element (4) is positioned such that fluid flowing through the first passage (10a) and the second passage (10b) converges to impinge on the temperature sensing element (4) at two different and diametrically opposite surfaces. The fluid is directed out of the device (100) through the outlet channel (2) subsequent to the impingement of the fluid on the temperature sensing element (4).

In an embodiment, the temperature sensing element (4) may be a self-heating source. The temperature sensing element (4) may be coupled to a control unit (7) and may be heated to required temperatures. The temperature sensing element (4) may also send out a signal corresponding to the temperature of the impinging fluid. In an embodiment, the concentric cylinders (3) may be made of any material including but not limited to metals, composites, polyethylene etc. The concentric cylinders (3) may be of a material that offers minimal resistance to the flow of fluid and may also be of a material that offers low thermal conductivity. Low thermal conductivity ensures that the temperature of the fluid that is being circulated through the concentric cylinders (3) remains un-affected.

In an embodiment, the plurality of passages (10) may be designed to include at least one of a converging profile and diverging profile. The at least one of the converging profiles and the diverging profile of the plurality of passages (10) may extend between the inlet port (1a) and the outlet port (2a) of the device (100). The at least one of the converging profiles and the diverging profile of the plurality of passages (10) may be configured to for regulate pressure of the fluid flowing through the plurality of passages (10). The profile of the plurality of passages (10) may be configured such that the speed of the fluid reaches an optimum range, where the fluid remains to impinge and contact the temperature sensing element (4) for a prolonged period of time. In an embodiment, at least one of the outer surfaces of the inner cylinder (3a) and the inner surface of the outer cylinder (3b) is configured with flow guiding members [not shown in figures]. The flow guiding members (not shown in figures) may be plate or flap like extensions that protrude outwardly from at least one of the outer surfaces of the inner cylinder (3a) and the inner surface of the outer cylinder (3b). The flow guiding members may assist in reduction of velocity of the flowing fluid or may enhance of the velocity of the flowing fluid. The flow guiding members in the first passage (10a) and the second passage (10b) may control the flow of fluid such that, maximum surface area of the temperature sensing element (4) is exposed to the flow of fluid, thereby increasing the sensitivity of the temperature sensing element. In an embodiment, the orientation of the flow guiding members may be varied by means of an actuator for either increasing or decreasing the velocity of fluid flowing in the plurality of passages (10). In an embodiment, the device (100) may be configured to utilize the Bernoulli's principle for controlling the fluid flow in the plurality of passages (10). An increase in speed of the fluid occurs simultaneously with a decrease in static pressure or a decrease in the fluid's potential energy. In an embodiment, the flow guiding members may be configured to decrease the fluid's potential energy for increasing the speed of the fluid or vice versa. In another embodiment, the plurality of passages (10) that facilitate the flow of the fluid may be pressurized by an external source and the pressure within the plurality of passages (10) may be varied for controlling the speed of the fluid. In an embodiment, the at least one of the converging profiles and the diverging profiles of the plurality of passages (10) may be configured to vary the pressure of the fluid flowing through the plurality of passages (10).

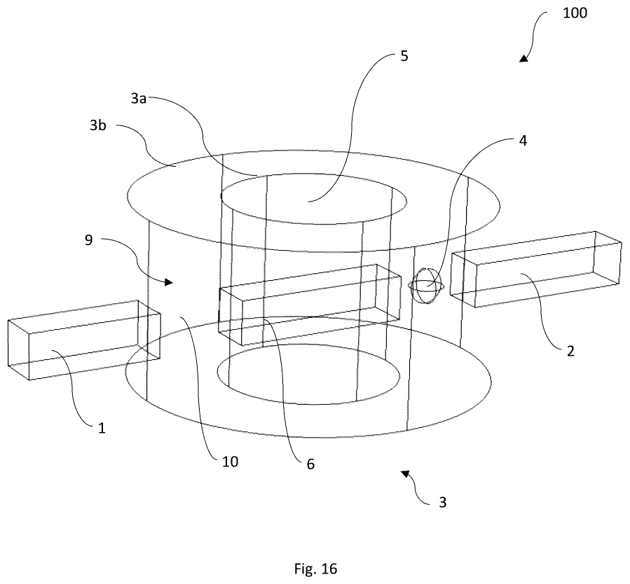

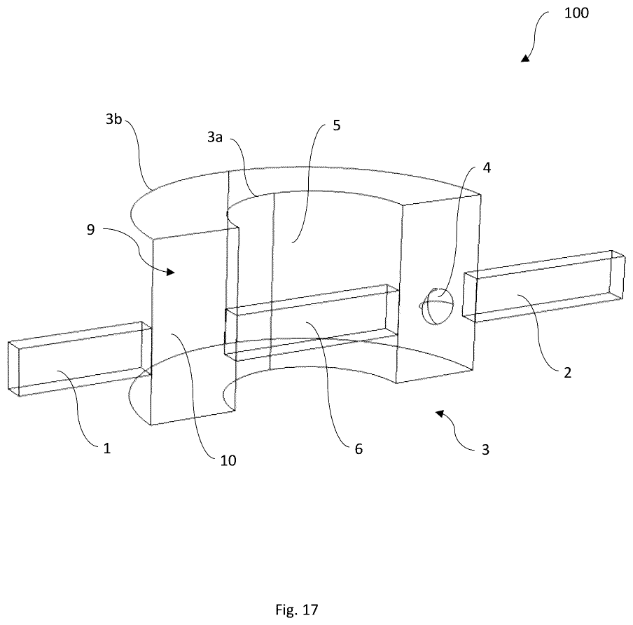

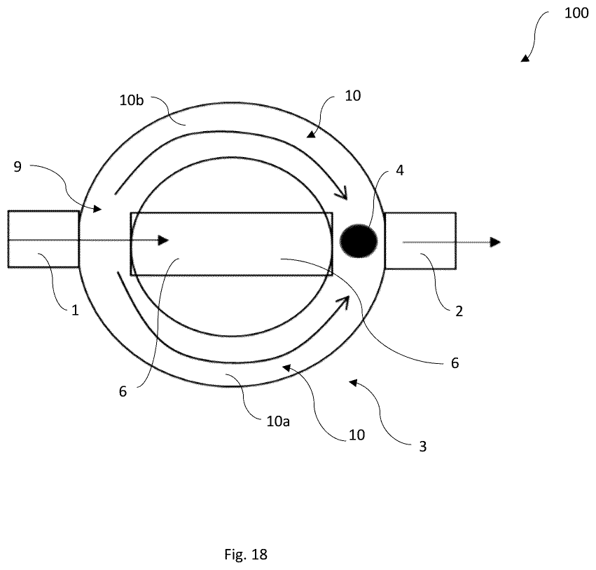

FIG. 16 and FIG. 17 illustrates a perspective view of an embodiment of the device (100). Similar to the above-described embodiment, the device (100) may also include the inner cylinder (3a) and the outer cylinder (3b) defining the chamber (9) with the plurality of passages (10). This embodiment of the device (100) may also include the inlet channel (1) and the outlet channel (2) configured to the outer cylinder (3b) in the above-described manner. The inlet channel (1) and the outlet channel (2) may be configured to extend from the chamber (9) in the diametrically opposite manner as described above. Further, the device (100) includes an auxiliary fluid flow passage (6). The auxiliary fluid flow passage (6) may interconnect either ends of the chamber (9). The auxiliary fluid flow passage (6) may be configured to lay along the same horizontal axis as that of the inlet channel (1) and the outlet channel (2). As seen from FIG. 17 and FIG. 18, the auxiliary fluid flow passage (6) may be configured to extend through the central annular passage (5) defined by the inner cylinder (3a). One end of the auxiliary fluid flow passage (6) may be configured proximal to the inlet channel (1) whereas, the other end of the auxiliary fluid flow passage (6) may be configured proximal to the outlet channel (2). The auxiliary fluid flow passage (6) may be configured to extend along the diameter of the inner cylinder (3a) and may be positioned at the rear of the temperature sensing element (4). Similar to the above-described embodiment, the temperature sensing element (4) may also be positioned at the point where the first passage (10a), the second passage (10b) and the auxiliary fluid flow passage (6) converges. The temperature sensing element (4) may be positioned proximal to the outlet channel (2). The fluid may initially enter the chamber (9) through the inlet channel (1). Subsequent to entering the chamber (9), the fluid flow gets divided into three different flow paths. The first flow path may be the fluid flowing through the auxiliary fluid flow passage (6). The second and the third flow paths may be the fluid flowing through the first passage (10a) and the second passage (10b) respectively. The fluid flowing through the first passage (10a), the second passage (10b) and the auxiliary fluid flow passage (6) converge at the point where the temperature sensing element (4) is positioned. Consequently, the fluid impinges the temperature sensing element at three different surfaces thereby, increasing the overall surface area of impingement of the temperature sensing element (4) that is exposed to the fluid. The fluid from the first passage (10a), the second passage (10b) and the auxiliary fluid flow passage (6) may be directed out of the device (100) after the fluid impinges the temperature sensing element (4).

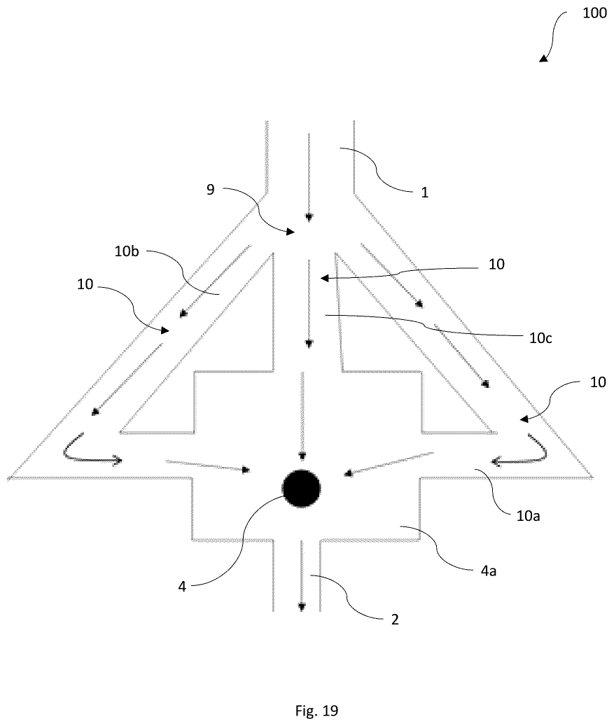

FIG. 19 illustrates a top view of another embodiment of the device (100). The device (100) may include an enclosure (4a) for accommodating the temperature sensing element (4). The enclosure (4a) may also be defined with multiple openings for facilitating the flow of fluid. In this particular embodiment, the enclosure (4a) may be a cube or a square shaped chamber. The shape of the enclosure (4a) must not be considered as a limitation since, the enclosure (4a) may be configured of any required shape. The device (100) in this particular embodiment also includes the inlet channel (1) and the outlet channel (2). The inlet channel (1) may be branched into three different channels. The inlet channel (1) may branch out into the first passage (10a), the second passage (10b) and the third passage (10c). The third passage (10c) may extend from the inlet channel (1) and may lie along the same axis as that of the inlet channel (1). The third passage (10c) may be fluidly coupled to a front end of the enclosure (4a). Further, the outlet channel (2) may extend from a rear end of the enclosure (4a) and the outlet channel (2) may be diametrically opposite to the third passage (10c). The first passage (10a) and the second passage (10b) that branch out from the inlet channel (1) extend angularly from the inlet channel and may further converge towards the enclosure (4a) as seem from FIG. 19. The first passage (10a) and the second passage (10b) may be fluidly coupled to the enclosure (4a) at either side of the enclosure (4a). Each of the plurality of passages (10), the inlet channel (1) and the outlet channel (2) may be spaced apart from each other at an angle of 90 degrees. The temperature sensing element (4) may be positioned within the enclosure (4a) at a central region of the enclosure (4a). The fluid flowing into the inlet channel (1) also branches into three different flow paths. The fluid from the inlet channel (1) may flow into the first passage (10a), the second passage (10b) and the third passage (10c). The fluid may subsequently converge into the enclosure (4a) to impinge on the temperature sensing element (4) at three different surfaces. Since the fluid impinges the temperature sensing element (4) at three different surfaces, a larger area of the temperature sensing clement (4) is exposed to the fluid.

Following paragraphs describe working of the device (100) and the system (200) according to the present disclosure. As described above with reference to the FIG. 11, the control unit (7) is communicatively coupled to the temperature sensing element (4) and the indication unit (8). The temperature sensing element (4) may initially be heated to the first temperature. Subsequently, the fluid may be circulated through the inlet channel (1) and the fluid may flow through the plurality of passages (10) to impinge on the temperature sensing element (4), as seen from FIG. 15, FIG. 18, and FIG. 19. The fluid that is initially circulated may herein be referred to the carrier fluid. The carrier fluid may be of the purest form with a high thermal conductivity. Since the thermal conductivity of the carrier fluid is high, the carrier fluid impinges the temperature sensing element (4), and the carrier fluid absorbs the heat from the temperature sensing element (4). The carrier fluid may initially cool the temperature sensing element (4) and the temperature of the temperature sensing element (4) may drop significantly below the initial first temperature to the second temperature. The temperature sensing element (4) may transmit a corresponding signal to the control unit (7). The control unit (7) may convert the received signal into the equivalent thermal signal. The thermal signal may further be indicated to the user through the indication unit (8) and the second temperature may be recorded. Further, the analyte fluid may be circulated to impinge the temperature sensing clement (4). The analyte fluid may herein be the fluid whose concentration is to be analyzed or determined. The analyte fluid is a mixture of fluids where the concentration of at least one fluid is to be determined. The thermal conductivity of the analyte fluid is lower due to the mixture of fluids. Consequently, when the analyte fluid is impinged on the temperature sensing element (4), the analyte fluid does not significantly cool the temperature sensing element (4) due to low thermal conductivity. Therefore, the temperature of the temperature sensing element (4) slightly increases to the third temperature. The third temperature is indicated through the control unit (7) and this increase in temperature directly corresponds to the concentration of the analyte fluid.

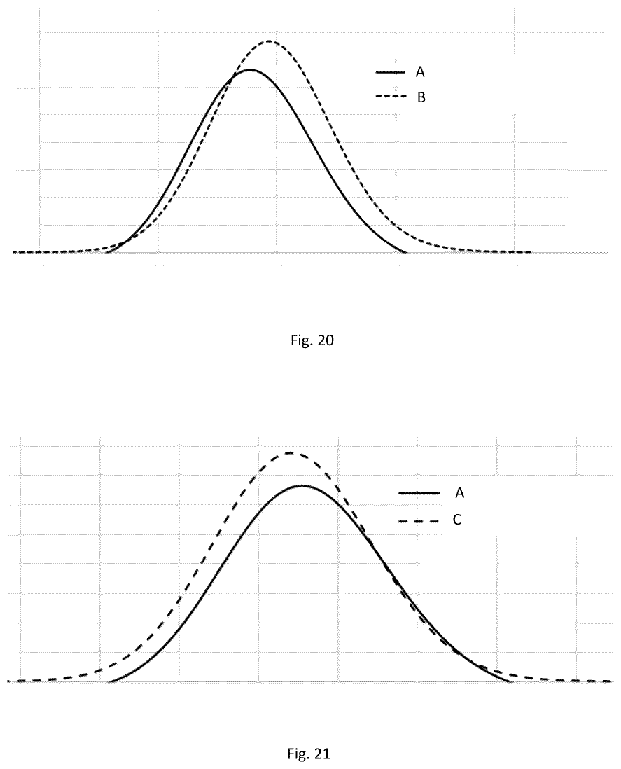

FIG. 20 illustrates a graphical representation of sensitivity of the thermal signal from the device (100) of FIG. 12 and FIG. 21 illustrates a graphical representation of the sensitivity of the thermal signal from the device (100) of FIG. 16. The FIG. 20 indicates the signal sensitivity of the device (100) from the present disclosure in dotted lines with a curve “B” whereas, the solid line with a curve “A” is indicative of the signal sensitivity from the conventional devices. Similarly, FIG. 21 indicates the signal sensitivity of the device (100) using three passages from the present disclosure in dotted lines with a curve “C” whereas, the solid line with the curve “A” is indicative of the signal sensitivity from the conventional devices. It is evident that the signal sensitivity is significantly greater in the device (100) of the present disclosure. Since the device (100) of the present disclosure is configured with the plurality of passages (10), the surface area of fluid impingement on the temperature sensing element (4) is drastically increased due to multiple fluid impingement surfaces on the temperature sensing clement (4). Consequently, the thermal signal sensitivity is also enhanced. Therefore, the accuracy of results in determining the concentration of a particular fluid in a mixture of fluids is significantly improved. Further, improved thermal signal sensitivity also enables the user to measure the concertation of a fluid with low volumes which subsequently, reduces the operational costs.

In an embodiment, the chamber (9) illustrated in the FIG. 12 may also be configured as the elliptic cylinder with the elliptical shape as illustrated in the FIG. 1. For instance, the chamber (9) in an embodiment may be of an elliptical shape and the chamber (9) may be defined with the plurality of passages (10) for the flow of the fluid. The elliptical shape of the chamber (9) may be defined by half of sum of the major axis (A) and the minor axis (B) of the chamber (9). The half of sum of the major axis (A) and the minor axis (B) ranges from 0.4 to 0.5 with a tolerance of +/−10% as illustrated above in the equation 1. The chamber (9) with the elliptical shape may be defined with the concentric cylinders (3) which define the plurality of passages (10). Similar to the above illustrated embodiment, the temperature sensing element (4) may be positioned proximal to the outlet channel (2) and the fluid may impinge the temperature sensing element (4) from two directions. In this embodiment, the plurality of passages (10) may also be configured with the elliptical shape since the chamber (9) is defined in the shape of the elliptic cylinder. In an embodiment, the device (100) illustrated in the FIG. 16 may also be defined as the elliptic cylinder. For instance, the chamber (9) may be of the elliptical shape and the plurality of passages (10) defined by the concentric cylinders (3) may also be elliptical in shape. Further, the auxiliary fluid flow passage (6) may extend through the elliptical shaped chamber (9) and the auxiliary fluid flow passage (6) may be configured to extend through the central annular passage (5) defined by the inner cylinder (3a). One end of the auxiliary fluid flow passage (6) may be configured proximal to the inlet channel (1) whereas, the other end of the auxiliary fluid flow passage (6) may be configured proximal to the outlet channel (2). In another embodiment, the device (100) from FIG. 12 and FIG. 16 may be defined with arcuate shaped sides. For instance, at least two opposing sides of the chamber (9) in the device from FIG. 12 and FIG. 16 may be defined with the convex shape. The convex shape of the at least two opposing sides of the chamber (9) may be defined with the pre-determined radius (R) and the thickness (t) of the chamber (9) may be measured along the longitudinal axis (A-A) of the chamber (9). Further, the at least two opposing sides with the convex shape may be defined by the ratio of the thickness (t) to the radius (R) of chamber (9). The ratio of the thickness (t) to the radius (R) of chamber (9) ranges from 0.15 to 0.20 with a tolerance of +/−20% as described above in the equation 2.

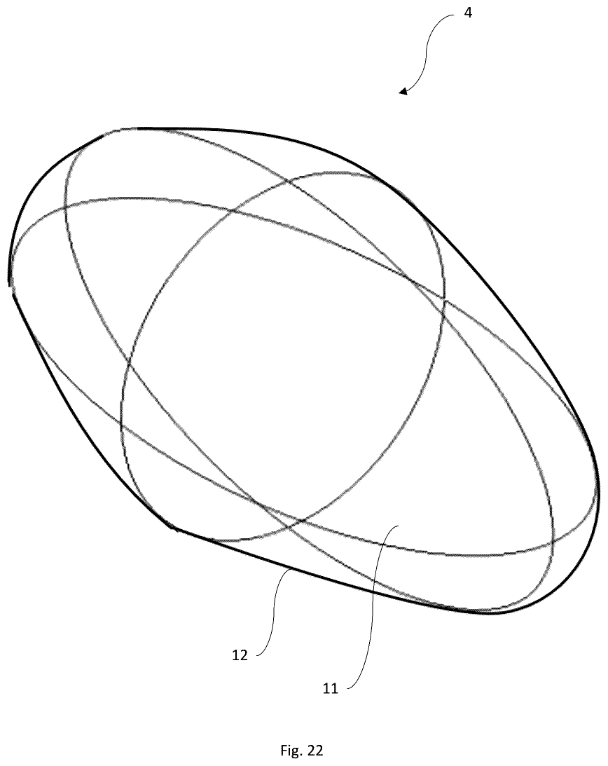

Further, the temperature sensing element (4) for the device (100) in the above illustrated embodiments is described below with greater detail. FIG. 22 illustrates a perspective view of the temperature sensing element (4). The temperature sensing element (4) may be defined by a body (11) and the body (11) in this particular embodiment may be in a shape of an ellipsoid. The body (11) of the temperature sensing element (4) may be configured to be hollow, and the body (11) may house a self-heating source. The self-heating source may be any known source, including but not limited to a metallic coil. The self-heating source may be configured to heat the body (11) by at least one of conduction and convection. The temperature sensing element (4) may be connected to the control unit (7) and may be heated to required temperatures. The control unit (7) may selectively operate the self-heating source to either increase the temperature or decrease the temperature of the body (11). The body (11) of the temperature sensing element (4) may be defined by an outer surface (12) and the outer surface (12) may be exposed to the fluid. The temperature sensing element (4) may be configured such that the fluid may impinge on the outer surface (12) of the body (11) and the fluid may either heat the temperature sensing element (4) or may absorb the heat from the body (11) of the temperature sensing element (4). The temperature sensing element (4) may also be configured with sensors that send out a signal to the control unit (7) corresponding to the temperature of the impinging fluid. In an embodiment, the temperature of the body (11) of the temperature sensing element (4) may be constantly monitored by the control unit (7) and any variations in the temperature of the body (11) subsequent to the impinging of the fluid on the body (11) may also be monitored and recorded by the control unit (7). With reference to FIG. 23, a fluid impingement area (14) on the outer surface (12) of the body (11) is indicated. The fluid impingement area (14) for the above ellipsoid shaped temperature sensing element (4) may range from about 28% to 32% of the total surface area and the overall area to volume ratio of the above temperature sensing element (4) may range from 44000 to 46000.

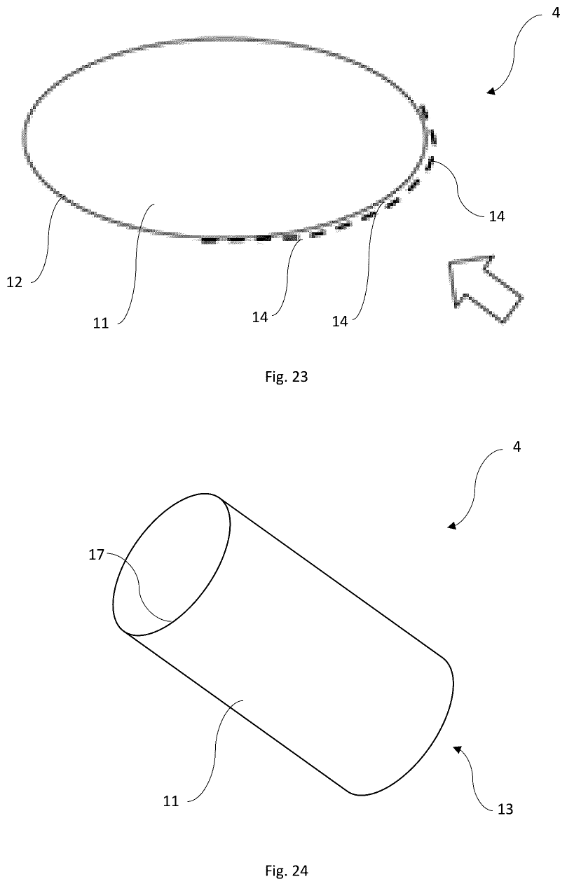

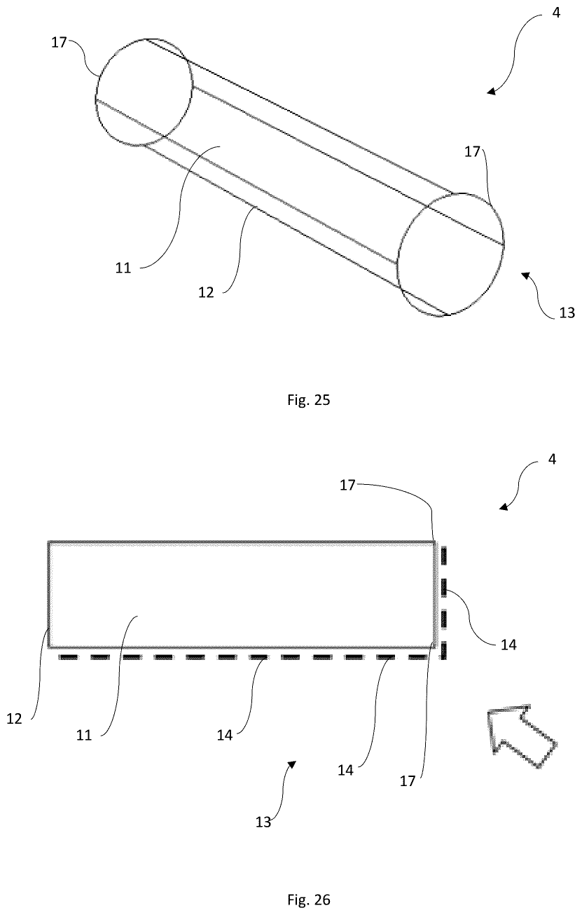

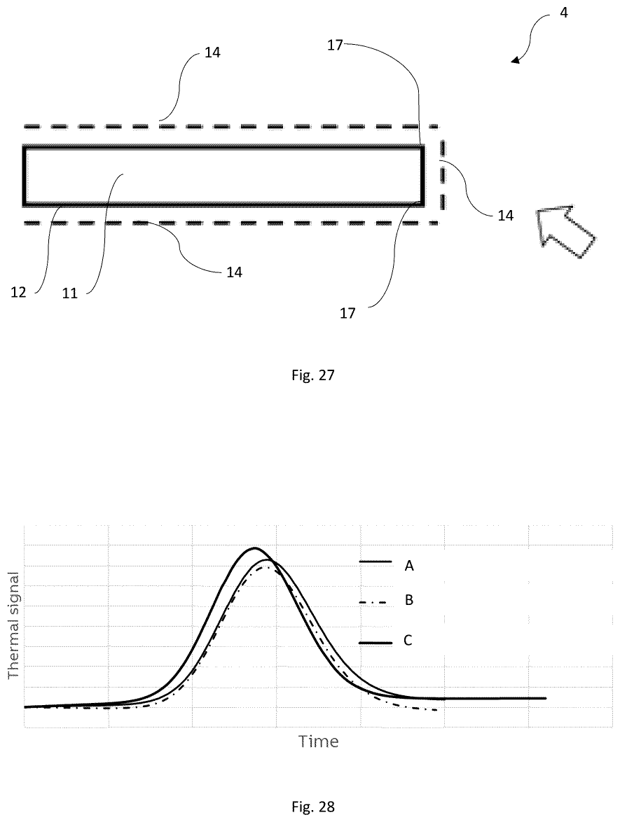

FIG. 24 and FIG. 25 illustrates a perspective view of an embodiment of the temperature sensing element (4). The temperature sensing element (4) in this embodiment may be in the shape of a cylinder. The temperature sensing element (4) in this embodiment may include a cylindrical body with a top surface and a bottom surface. The temperature sensing element (4) may be defined with edges (17) (hereinafter referred to as “edge”) along a region where the cylindrical body meets with the top surface and the bottom surface. Similar to the above embodiment, the temperature sensing element (4) may also be defined by a body (11) and may include the self-heating source housed within the temperature sensing element (4). The temperature sensing element (4) may be connected to the control unit (7) and the control unit (7) may selectively operate the self-heating source to either increase the temperature or decrease the temperature of the body (11). The body (11) of the temperature sensing element (4) may also be defined by the outer surface (12) and the outer surface (12) may be exposed to the fluid. As seen from FIG. 26, the temperature sensing element (4) may be positioned such that the fluid impinges on the edge (17) of the body (11). The fluid may directly impinge on the edge (17) of the body (11), the top surface of the body (11) and the cylindrical portion of the body (11). With reference to the FIG. 27, the temperature sensing element (4) may also be configured such that the fluid impinges on at least one of the top surfaces or the bottom surface. Subsequently, the fluid may traverse over the outer surface (12) of the temperature sensing element (4) as indicated by the dotted lines in the FIG. 27. The fluid impinging on the outer surface (12) of the body (11) may either heat the temperature sensing clement (4) or may absorb the heat from the body (11) of the temperature sensing element (4). The temperature sensing element (4) may be configured to send out the signal to the control unit (7) corresponding to the temperature of the impinging fluid. The fluid impingement area (14) for the above cylindrical shaped temperature sensing element (4) may range from about 70% to 80% of the total surface area and the overall area to volume ratio of the above temperature sensing element (4) may range from 37800 to 39800. Further, the ratio of direct impact area of the fluid on the temperature sensing element (4) to total area of the temperature sensing element (4) may range from 0.5 to 0.8.

FIG. 28 illustrates a graphical representation of the sensitivity of the temperature sensing element (4) with the shape of the ellipsoid and the cylinder. The curve “A” represents the sensitivity of a conventional temperature sensing element (4). The curve “B” and the curve “C” represents the sensitivity of the temperature sensing element (4) with the shape of the ellipsoid and the cylinder, respectively as illustrated above. It is evident from the FIG. 7 that the signal sensitivity of the temperature sensing clement (4) with the cylindrical shape is significantly greater than the sensitivity of the temperature sensing clement (4) with the ellipsoid shape or the conventional spherical shape.

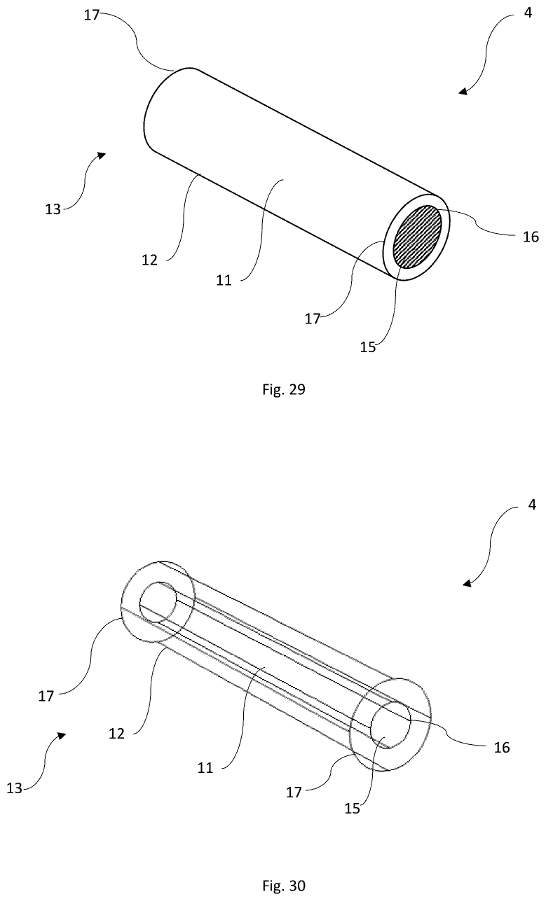

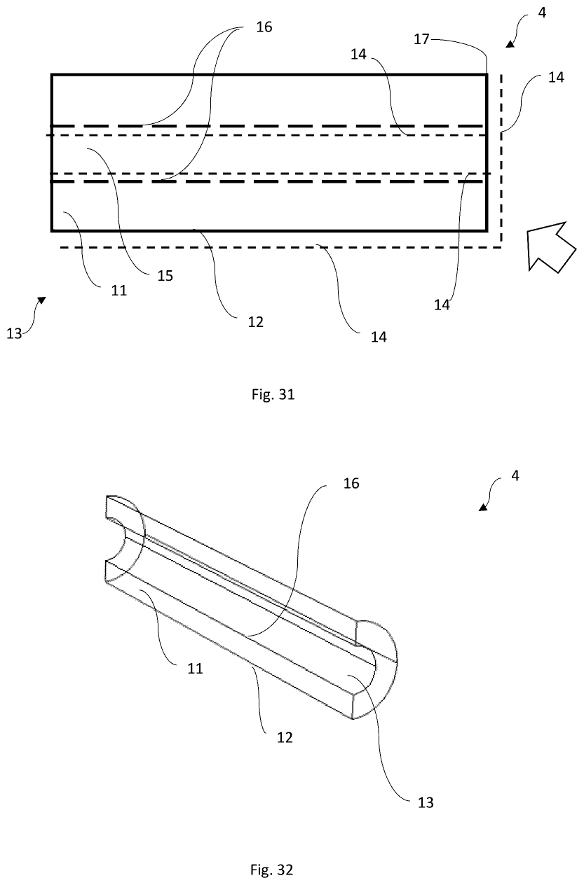

FIG. 29 and FIG. 30 illustrates a perspective view of another embodiment of the temperature sensing element (4). The temperature sensing element (4) in this embodiment may be a body (11) in the shape of a cylinder with a through passage (15). The through passage (15) may extend along a central horizontal axis of the temperature sensing element (4). Further, the through passage (15) may be defined by an inner surface (16). Similar to the above-mentioned embodiment, the temperature sensing element (4) in this embodiment may also be defined by cylindrical wall with the top surface and the bottom surface. The temperature sensing element (4) may be defined with edges (17) (hereinafter referred to as “edge”) along a region where the cylindrical wall meets with the top surface and the bottom surface. Similar to the above embodiment, the temperature sensing element (4) may also be defined by the body (11) and may include the self-heating source housed within the temperature sensing element (4). The temperature sensing element (4) may be connected to the control unit (7) and the control unit (7) may selectively operate the self-heating source to either increase the temperature or decrease the temperature of the body (11). The body (11) of the temperature sensing element (4) may also be defined by the outer surface (12) and the outer surface (12) may be exposed to the fluid. With reference to the FIG. 31, the temperature sensing element (4) may be configured such that the fluid directly impinges on the cylindrical wall and at least one of the top surfaces or the bottom surface. Subsequently, the fluid may traverse over the outer surface (12) of the temperature sensing element (4) as indicated by the dotted lines in the FIG. 31. The fluid may also enter the through passage (15) and may traverse over the inner surface (16) of the through passage (15). Thus, a fluid impingement area (14) for the above embodiment may lie along the inner surface (16) of the through passage (15), the cylindrical wall of the temperature sensing element (4) and at least one of the top surfaces and the bottom surface of the temperature sensing element (4). The fluid impinging on the body (11) may either heat the temperature sensing element (4) or may absorb the heat from the body (11) of the temperature sensing element (4). The temperature sensing element (4) may send out the signal to the control unit (7) corresponding to the temperature of the impinging fluid. The fluid impingement area (14) for the above cylindrical shaped temperature sensing element (4) with the through passage (15) may range from about 70% to 80% of the total surface area and the overall area to volume ratio of the above temperature sensing element (4) may range from 37800 to 39800.

In another embodiment, the fluid impingement area (14) for the above cylindrical shaped temperature sensing element (4) with the through passage (15) may range from about 70% to 80% of the total surface area with the overall area to volume ratio of the above temperature sensing element (4) ranging from 111000 to 113000. The range of direct impact area of the fluid on the temperature sensing element (4) to total area of the temperature sensing element (4) for the above cylindrical shaped temperature sensing element (4) with the through passage (15) may range from 0.5 to 0.8.

In another embodiment, the fluid impingement area (14) for the temperature sensing element (4) may range from about 70% to 80% of the total surface area with the overall area to volume ratio of the above temperature sensing element (4) ranging from 30000 to 300000.

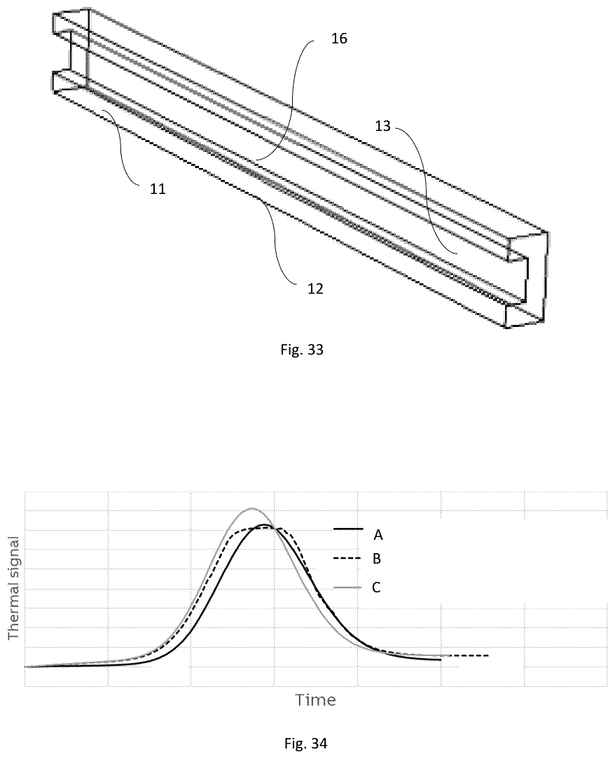

FIG. 32 and FIG. 33 illustrates a perspective view of another embodiment of the temperature sensing element (4). The temperature sensing element (4) may be defined by a body (11) with a semi cylindrical shape. As seen from FIG. 32, the semi cylindrical shaped temperature sensing element (4) may be defined by a central cutout (13). The cutout (13) may extend throughout the length of the temperature sensing element (4) and an inner surface (16) may be defined by the cutout (13). In an embodiment, the cutout (13) may be defined by multiple apertures along the inner surface (16) of the cutout (13) and the multiple apertures increase the fluid impingement area (14). FIG. 33 illustrates the body (11) of the temperature sensing clement (4) in the shape of a semi hollow cuboid. The cuboid shaped temperature sensing element (4) may be defined with a cutout (13) on one of the sides. The cutout (13) in this embodiment may also be defined by the inner surface (16). In an embodiment, the cutout (13) may be defined by multiple apertures along the inner surface (16) for increasing the fluid impingement area (14).

FIG. 34 illustrates a graphical representation of the sensitivity of the temperature sensing element (4) with the shape of the hollow cylinder and the conventional spherical shape. The curve “A” represents the sensitivity of a conventional temperature sensing element (4). The curve “B” represents the sensitivity of the temperature sensing element (4) in the shape of the cylinder with the through passage (15) where, the overall area to volume ratio of the above temperature sensing element (4) may range from 37800 to 39800. The curve “C” represents the sensitivity of the temperature sensing element (4) in the shape of the cylinder with the through passage (15) where, the overall area to volume ratio of the above temperature sensing element (4) may range from 111000 to 113000. It is evident from the FIG. 34 that the signal sensitivity of the temperature sensing element (4) is significantly greater for the curve “C” where, the area to volume ratio of the above temperature sensing element (4) may range from 111000 to 113000. The signal sensitivity for the above configuration of the temperature sensing element (4) is significantly greater than the sensitivity for the temperature sensing element (4) with the conventional spherical shape or the cylindrical shape with the through passage (15) where overall area to volume ratio is from 37800 to 39800.

In an embodiment, the thermal signal from the temperature sensing element (4) of the present disclosure is enhanced since, the fluid impingement area (14) on the temperature sensing element (4) is increased to around 70% to 80% and the area to volume ratio of the temperature sensing element (4) is in the range of 111000 to 113000. Since the sensitivity of the signal from the temperature sensing element (4) is improved, the accuracy of results in determining the concentration of a particular fluid in a mixture of fluids is significantly improved. Further, improved thermal signal sensitivity also enables the user to measure the concertation of a fluid with low volumes which subsequently, reduces the operational costs.

In an embodiment, the elliptical shape of the chamber (9) and the convex shape of the at least two opposing sides (21, 22 and/or 23, 24) ensures optimum velocity of fluid impingement on the temperature sensing element (4).

In an embodiment, the temperature sensing element (4) illustrated in the FIG. 23 to FIG. 33 may be configured to the device (100) illustrated in the FIG. 1 to FIG. 19. For instance, the temperature sensing element (4) illustrated in the FIG. 29 may be positioned inside the chamber (9) in the device (100) form the FIG. 1. The elliptical shaped chamber (9) may accommodate the temperature sensing element (4) from the FIG. 29 that is shaped in the form of the cylinder with the through passage (15). The velocity of the fluid entering the chamber (9) is optimized due to the elliptical shape of the chamber (9) and the fluid impingement area is maximized due to the cylindrical shape with the through passage (15) of the temperature sensing element (4). In an embodiment, the temperature sensing element (4) that is accommodated in the chamber (9) from the device (100) in the FIG. 1, may be of the elliptical shape as illustrated in the FIG. 22. The shape of the temperature sensing element (4) that is accommodated in the chamber (9) may also in the shape of the cylinder as illustrated from the FIG. 24.

In an embodiment, the temperature sensing element (4) illustrated in the FIG. 29 may also be positioned proximal to the outlet channel (2) in the device (100) from the FIG. 12. In an embodiment, the temperature sensing element (4) illustrated in the FIG. 29 may also be positioned in the device (100) from the FIG. 16 where, the fluid impinges from three different direction onto the temperature sensing element (4). The temperature sensing element (4) that is positioned in the device (100) from the FIG. 12 and the FIG. 16 may be in the shape of the ellipse, the cylinder, the cylinder with the through passage (15) as illustrated in the FIG. 22, the FIG. 24, and the FIG. 29 respectively. In an embodiment, the chamber (9) which is of the elliptical shape with the convex shape defined on the at least two opposing sides as seen from the FIG. 5 and FIG. 8 may also accommodate the temperature sensing element (4) that is in one of the shapes of the ellipse, the cylinder, the cylinder with the through passage (15) as illustrated in the FIG. 22, the FIG. 24, and the FIG. 29 respectively.

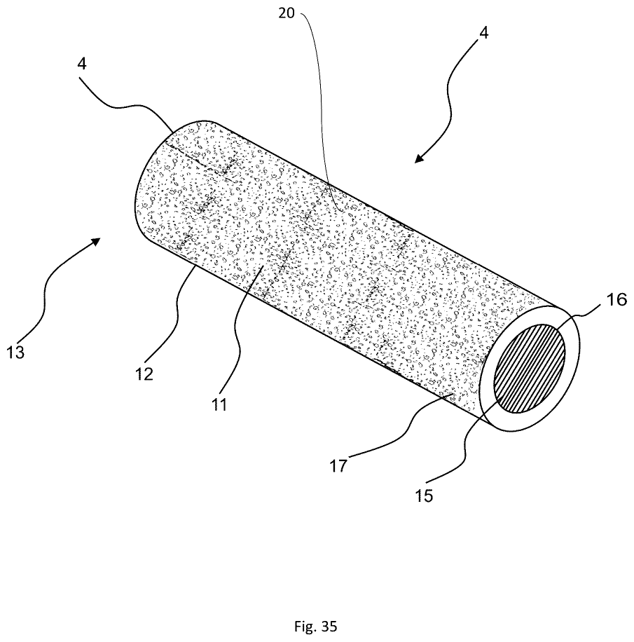

In another embodiment, the inner surface (16) of the through passage (15) may be defined by multiple pores (20) as seen from FIG. 35. The multiple pores (20) may increase the fluid impingement area (14) and subsequently improve the sensitivity of the thermal signal form the temperature sensing element (4).

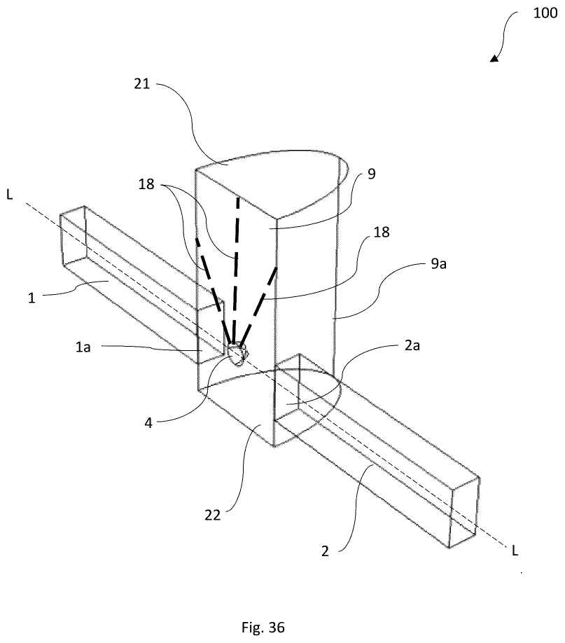

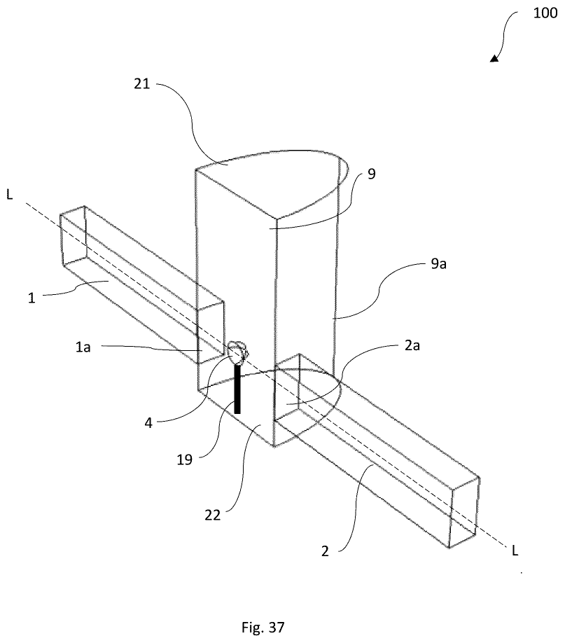

With reference to FIG. 36, the temperature sensing element (4) may be suspended from an inner surface of the chamber (9) by at least one tethers (18) [hereinafter referred to as the tether]. In this exemplary embodiment, one end of the tether (18) is fixedly connected to the inner surface of the chamber (9) and the other end of the tether (18) is fixedly connected to the temperature sensing element (4). The tension in the tethers (18) may be configured such that the temperature sensing element (4) is suspended in the chamber (9) without any movement. Further, the length of the tethers (18) may be configured such that the temperature sensing element (4) is configured to lie along the lateral axis (L-L). In an embodiment, a support structure (19) may be configured to extend from a bottom wall of the chamber (9a) as seen from FIG. 37. The support structure (19) may be configured to support the temperature sensing element (4) and the height of the support structure (19) may be configured such that the temperature sensing element (4) positioned on the support structure (19), may lie along the lateral axis (A-A). In an embodiment, the support structure (19) and the tethers (18) may be used together to support the temperature sensing element (4) inside the chamber (9a).

In another embodiment, the device (100) may be defined with the elliptical shape and the elliptical shape of the chamber (9) may be defined by half of sum of the major axis (A) and the minor axis (B) of the chamber (9). The half of sum of the major axis (A) and the minor axis (B) ranges from 0.4 to 0.5 with a tolerance of +/−10% as illustrated above in the equation 1. Further, at least two opposing sides of the chamber (9) may be defined with the convex shape. The convex shape may be defined by the ratio of the thickness (t) to the radius (R) of chamber (9). The ratio of the thickness (t) to the radius (R) of chamber (9) ranges from 0.15 to 0.20 with a tolerance of +/−20% as described above in the equation 2. The device (100) may also include the plurality of passages (10) with auxiliary fluid flow passage (6). The above illustrated device (100) may further accommodate the temperature sensing element (4) which is in the shape of the cylinder (4) with the through passage (15). The plurality of passages (10) with the auxiliary fluid flow passage (6) and the temperature sensing element (4) which is in the shape of the cylinder with the through passage (15) may provide a configuration where the area of fluid impingement on the temperature sensing element (4) is maximized. Further, the elliptical shape of the chamber (9) with convex shape on at least two opposing sides also optimizes the velocity of the fluid that impinges the temperature sensing element (4). The above-illustrated embodiment may be considered as the most efficient embodiment.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERRAL NUMERALS

| Description | Referral numerals |
|---|---|
| Inlet channel | 1 |
| Inlet port | 1a |
| Outlet channel | 2 |
| Outlet port | 2a |
| Concentric cylinders | 3 |
| Inner cylinder | 3a |
| Outer cylinder | 3b |
| Temperature sensing element | 4 |
| Enclosure | 4a |
| Central annular passage | 5 |
| Auxiliary fluid flow passage | 6 |
| Control unit | 7 |
| Indication unit | 8 |
| Chamber | 9 |
| Wall | 9a |
| Plurality of passages | 10 |
| First passage | 10a |
| Second passage | 10b |
| Third passage | 10c |
| Body | 11 |
| Outer surface of the body | 12 |
| Cutout | 13 |
| Fluid impingement area | 14 |
| Through passage | 15 |
| Inner surface of the temperature sensing | 16 |
| element | |
| Edge | 17 |
| Tethers | 18 |
| Support structure | 19 |
| Pores | 20 |
| Top cover | 21 |
| Bottom cover | 22 |
| Front cover | 23 |
| Rear cover | 24 |
| Major axis | A |
| Minor axis | B |
| Thermal conductivity detector device | 100 |
| System | 200 |

What is claimed is:

1. A thermal conductivity detector device, comprising:
a chamber defined within the device, the chamber having an inlet channel and an outlet channel;
wherein the chamber is of an elliptical shape;
wherein, at least two opposing sides of the chamber are defined in an arcuate shape and the chamber is defined in the shape of a venturi; and
a temperature sensing element positioned within the chamber;
wherein the temperature sensing element is configured to determine the temperature of a fluid flowing through the chamber from the inlet channel towards the outlet channel.

2. The device of claim 1, wherein the arcuate shape of the at least two opposing sides of the chamber is of a pre-determined radius.

3. The device of claim 1, wherein the chamber is defined by a major axis and a minor axis, and wherein half of a sum of the major axis and the minor axis ranges from 0.4 to 0.5.

4. The device of claim 1, wherein the chamber is defined with a thickness measured along a longitudinal axis of the chamber.

5. The device of claim 4, wherein the ratio of the thickness to the pre-determined radius ranges from 0.15 to 0.20.

6. The device of claim 1, wherein each of the inlet channel and the outlet channel are defined on at least one of the same sides of the chamber, two opposing sides of the chamber and two diagonal sides of the chamber.

7. The device of claim 1, wherein all sides of the chamber are defined with the arcuate shape.

8. The device of claim 1, wherein the temperature sensing element is suspended from an inner surface of the chamber.

9. The device of claim 1, wherein the temperature sensing element is one of a bead with a cylindrical shape, an elliptical shape, a cylindrical shape with a through passage.

10. A system for estimating concentration of a fluid, comprising:
a thermal conductivity detector device, the device comprising a chamber defined with an inlet channel and an outlet channel;
wherein the chamber is of an elliptical shape; and
wherein at least two opposing sides of the chamber are defined in an arcuate shape and the chamber is defined in a shape of a venturi;
a temperature sensing element positioned within the chamber;
a control unit that is communicatively coupled to the temperature sensing element, the control unit configured to:
receive a signal from the temperature sensing element corresponding to the temperature of the fluid flowing through the chamber from the inlet channel to the outlet channel; and
indicate the temperature of the fluid through an indication unit;
wherein the temperature of the fluid corresponds to the concentration of the fluid.

11. A method for estimating concentration of a fluid from a system, comprising:
channeling fluid from an inlet channel to an outlet channel of a chamber, wherein the chamber is of an elliptical shape and wherein at least two opposing sides of the chamber are defined in an arcuate shape such that the fluid impinges a temperature sensing element positioned in the chamber;
receiving by a control unit a signal from the temperature sensing element; and
indicating by an indication unit connected to the control unit the temperature of the fluid flowing through the chamber;
wherein the temperature of the fluid corresponds to the concentration of the fluid.
